(19) United States
(12) Patent Application Publication
(10) Pub. No.: US 2007/0185129 A1
(43) Pub. Date: Aug. 9, 2007

(54) ACID ADDITION SALTS OF THIENOPYRANCARBOXAMIDE DERIVATIVES

(75) Inventors: Amedeo Leonardi, Milan (IT); Gianni Motta, Barlassina (IT)

Correspondence Address: DARBY & DARBY P.C., P.O. BOX 770, Church Street Station, New York, NY, 10008-0770, US

(73) Assignee: Recordati Ireland Limited, Ringaskiddy (IE)

(21) Appl. No.: 11/671,350

(22) Filed: Feb. 5, 2007

Related U.S. Application Data
(60) Provisional application No. 60/764,824, filed on Feb. 3, 2006.

(51) Int. Cl. A61K 31/496; C07D 495/02

(52) U.S. Cl. 514/252.13; 544/375

(57) ABSTRACT

The invention relates to novel addition salts of N-{3-[4-(2-Methoxyphenyl)-1-piperazinyl]propyl}-7-oxo-5-trifluoromethyl-7H-thieno[3,2-b]pyran-3-carboxamide, comprising N-{3-[4-(2-Methoxyphenyl)-1-piperazinyl]propyl}-7-oxo-5-trifluoromethyl-7H-thieno[3,2-b]pyran-3-carboxamide and an acid counterion wherein the acid counterion is selected from the group consisting of: (i) inorganic acids and (ii) sulfonic acids.

### CROSS REFERENCE TO PRIOR APPLICATIONS

This application claims benefit under 35 U.S.C. 119(e) to U.S. Provisional Application No. 60/764,824 filed Feb. 3, 2006 which is incorporated by reference herein in its entirety.

### FIELD OF THE INVENTION

The invention is directed to novel crystalline acid addition salts of N-{3-[4-(2-methoxyphenyl)-1-piperazinyl]prop yl}-7-oxo-5-trifluoromethyl-7H-thieno[3,2-b]pyran-3-carboxamide and pharmaceutical compositions thereof. The invention is further directed to the use of said compounds and compositions for treating obstructive syndromes of the lower urinary tract.

### BACKGROUND OF THE INVENTION

U.S. Pat. Nos. 6,387,909, 6,306,861 and 6,486,163, all of which are incorporated herein by reference, disclose the preparation of thienopyranecarboxamide derivatives having the general formula (I):. 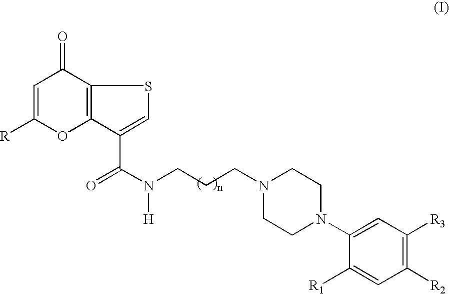
wherein
R is an aryl, cycloalkyl or polyhaloalkyl group;
R₁ is chosen from the group consisting of alkyl, alkoxy, polyfluoroalkoxy, hydroxy and trifluoromethanesulfonyloxy;
each of R₂ and R₃ are independently selected from the group consisting of a hydrogen, halogen, alkoxy and polyfluoroalkoxy group; and
n is 0, 1 or 2.

Compounds having the general formula (I) have high affinity for the α₁ adrenergic receptor, with selectivity toward the α₁ adrenergic receptor as compared to the 5-HT_{1A} receptor. Within the α₁ adrenergic family, compounds of formula (I) typically exhibit further selectivity for the α₁ₐ receptor compared to α_{1b} and α_{1d} receptors.

One compound in the aforementioned U.S. patents is the free base of N-{3-[4-(2-methoxyphenyl)-1-piperazinyl]propyl}-7-oxo-5-trifluoromethyl-7H-thieno-[3,2-b]pyran-3-carboxamide, having the structure shown in formula (II):

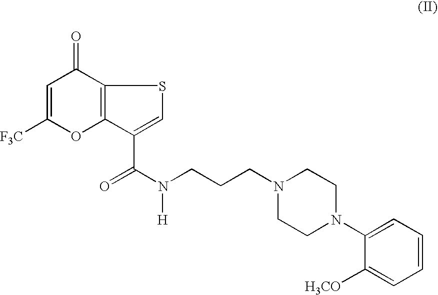

N-{3-[4-(2-methoxyphenyl)-1-piperazinyl]propyl}-7-oxo-5-trifluoromethyl-7H-thieno[3,2-b]pyran-3-carboxamide (the compound of formula II; prepared according to the methods of U.S. Pat. No. 6,387,909) has a high affinity for the α₁ₐ receptor and exhibits approximately a 45-fold selectivity for the recombinant human α₁ₐ receptor relative to the recombinant human 5-HT_{1A} receptor. The compound also exhibits selectivity among the α₁ receptors. Hence, the compound exhibits about a 40-fold selectivity for α₁ₐ receptor, as compared to the α_{1b} receptor and about a 6-fold selectivity for α₁ₐ receptor as compared to the α_{1d} receptor. The compound also demonstrates functional antagonism of the α_{1L} receptor, as measured in an isolated rabbit aorta model system.

The adrenergic antagonistic activity and high uroselectivity of N-{3-[4-(2-methoxyphenyl)-1-piperazinyl]propyl}-7-oxo-5-trifluoromethyl-7H-thieno[3,2-b]pyran-3-carboxamide makes it a useful agent for acting on body tissues rich in α₁ adrenergic receptors, such as the prostate gland and the urethra. Accordingly, the compound is a useful therapeutic agent for the treatment of, for example, micturition problems associated with obstructive disorders of the lower urinary tract, including but not limited to benign prostatic hypertrophy (BPH).

Although the pharmacological properties of N-{3-[4-(2-methoxyphenyl)-1-piperazinyl]propyl}-7-oxo-5-trifluoromethyl-7H-thieno[3,2-b]pyran-3-carboxamide make the compound a desirable agent for treatment of obstructive disorders of the lower urinary tract, the free base form of the compound described in the prior art has disadvantages in preparation and possesses physical properties that make it difficult to adapt for commercial use and formulation into pharmaceutical compositions. The prior art method of producing the free base, for example, required purification by flash chromatography and had an overall yield of only about 30%. Additionally, the free base may exist as different polymorphs, each of which may have distinct physical properties, which further contributes to production difficulties. The free base also exhibits poor water solubility, heat instability, and is difficult to crystallize. These factors make the free base poorly suited for industrial manufacturing and formulation into pharmaceutical compositions.

Accordingly, there is a need for additional forms of N-{3-[4-(2-methoxyphenyl)-1-piperazinyl]propyl}-7-oxo-5-trifluoromethyl-7H-thieno[3,2-b]pyran-3-carboxamide that exhibit improved production, and physical and pharmaceutical characteristics, as compared to the prior art free base. The present inventors have discovered that acid addition salts of N-{3-[4-(2-methoxyphenyl)-1-piperazinyl]propyl}-7-oxo-5-trifluoromethyl-7H-thieno[3,2-b]pyran-3-carboxamide overcome at least some of the disadvantages of the free base. Although some of the acid addition salts described herein are produced as a mixture of multiple polymorphs, the monomethanesulfonate salt has the advantage of being easily isolated as a single polymorph with improved water solubility compared to the free base.

### SUMMARY OF THE INVENTION

The invention provides novel acid addition salts of N-{3-[4-(2-methoxyphenyl)-1-piperazinyl]propyl}-7-oxo-5-trifluoromethyl-7H-thieno[3,2-b]pyran-3-carboxamide, pharmaceutical compositions thereof, and methods of using such acid addition salts to treat obstructive syndromes of the lower urinary tract, including benign prostatic hyperplasia (BPH), and lower urinary tract symptoms (LUTS).

In one embodiment, the invention provides a compound selected from the group consisting of an inorganic acid, sulfonic acid, monocarboxylic acid, dicarboxylic acid, tricarboxylic acid, and aromatic sulfonamide acid addition salts of N-{3-[4-(2-methoxyphenyl)-1-piperazinyl]propyl}-7-oxo-5-trifluoromethyl-7H-thieno[3,2-b]pyran-3-carboxamide.

In certain preferred embodiments the invention provides a crystalline acid addition salt of N-{3-[4-(2-methoxyphenyl)-1-piperazinyl]propyl}-7-oxo-5-trifluoromethyl-7H-thieno[3,2-b]pyran-3-carboxamide selected from the group consisting of crystalline mesylate, crystalline hydrochloride, crystalline hydrobromide, and crystalline besylate salts. The crystalline acid addition salts may be present as a single crystal form (i.e., a single polymorph), or as mixtures of more than one crystal form (i.e., a mixture of polymorphs).

In another embodiment, the invention provides the aforementioned acid addition salts of N-{3-[4-(2-methoxyphenyl)-1-piperazinyl]propyl}-7-oxo-5-trifluoromethyl-7H-thieno[3,2-b]pyran-3-carboxamide in solvated and hydrated forms, particularly mono- and dehydrates and solvates and more particularly mono- and dimethanolates.

In yet another embodiment methods are provided for the preparation of the aforementioned acid addition salts of N-{3-[4-(2-methoxyphenyl)-1-piperazinyl]propyl}-7-oxo-5-trifluoromethyl-7H-thieno[3,2-b]pyran-3-carboxamide, including both amorphous and crystalline acid addition salts, utilizing the free base of N-{3-[4-(2-methoxyphenyl)-1-piperazinyl]propyl}-7-oxo-5-trifluoromethyl-7H-thieno[3,2-b]pyran-3-carboxamide as the starting material.

Also provided are pharmaceutical compositions comprising an acid addition salt of N-{3-[4-(2-methoxyphenyl)-1-piperazinyl]propyl}-7-oxo-5-trifluoromethyl-7H-thieno[3,2-b]pyran-3-carboxamide and a pharmaceutically acceptable excipient, e.g., a carrier, diluent, flavorant, sweetener, preservative, dye, binder, suspending agent, dispersing agent, colorant, disintegrant, lubricant, plasticizer, or edible oil.

These and other aspects of the invention will be apparent to those of ordinary skill in the art in light of the present description, claims and figures.

### BRIEF DESCRIPTION OF THE DRAWINGS

FIG. 1 is a DSC profile for recrystallized (acetone/Et₂O) N-{3-[4-(2-methoxyphenyl)-1-piperazinyl]propyl}-7-oxo-5-trifluoromethyl-7H-thieno[3,2-b]pyran-3-carboxamide mesylate salt, melting at 186° C. (DSC).

FIG. 2 is a DSC profile for N-{3-[4-(2-methoxyphenyl)-1-piperazinyl]propyl}-7-oxo-5-trifluoromethyl-7H-thieno[3,2-b]pyran-3-carboxamide hydrochloride salt formed by evaporation of the mother liquor (MeOH/Et₂O).

FIG. 3 is a DSC profile for N-{3-[4-(2-methoxyphenyl)-1-piperazinyl]propyl}-7-oxo-5-trifluoromethyl-7H-thieno[3,2-b]pyran-3-carboxamide hydrochloride salt, obtained after dissolution in CH₂Cl₂/EtOH, evaporation to dryness and crushed in Et₂O.

FIG. 4 is a DSC profile for recrystallized (EtOH) N-{3-[4-(2-methoxyphenyl)-1-piperazinyl]propyl}-7-oxo-5-trifluoromethyl-7H-thieno[3,2-b]pyran-3-carboxamide hydrobromide salt.

FIG. 5 is a DSC profile for crystallized (EtOH/Et₂O) N-{3-[4-(2-methoxyphenyl)-1-piperazinyl]propyl}-7-oxo-5-trifluoromethyl-7H-thieno[3,2-b]pyran-3-carboxamide besylate salt.

FIG. 6 is an IR spectrum for N-{3-[4-(2-methoxyphenyl)-1-piperazinyl]propyl}-7-oxo-5-trifluoromethyl-7H-thieno[3,2-b]pyran-3-carboxamide mesylate salt prepared from the free base and methanesulfonic acid in acetone, melting at 186° C. (DSC).

FIG. 7 is an XRD spectrum for N-{3-[4-(2-methoxyphenyl)-1-piperazinyl]propyl}-7-oxo-5-trifluoromethyl-7H-thieno[3,2-b]pyran-3-carboxamide mesylate m.p. 186° C.

### DETAILED DESCRIPTION OF THE INVENTION

As used herein, the following terms are defined as follows:

The term “amorphous” refers to compounds in the solid state and having no substantial crystal lattice structure. When analyzed by differential scanning calorimetry (DSC), amorphous compounds provide broad exothermic transitions, defined as glass transitions, in place of the sharper exothermic peaks characteristic of crystalline compounds. Amorphous solids also show no characteristic peaks in X-Ray diffraction.

The term “crystalline” refers to crystals of a solid compound having a melting point and X-ray diffraction pattern characteristic of a crystalline form. When analyzed by DSC, crystalline compounds show sharp exothermic peaks characteristic of the melting point range for its respective crystalline form.

The terms “polymorphic” or “polymorphism” refer to a property of a compound to exist in one or more distinct crystal forms, each with different structures. Polymorphic crystalline forms (i.e., “polymorphs”) can be detected directly by crystallographic techniques or indirectly by assessment of differences in physical and/or chemical properties associated with each particular polymorph. For example, a crystal that contains more than one polymorph can show more than one distinct melting point in DSC.

The term “single crystal form” or “single crystalline form” refers to a crystalline material that contains a single polymorph. It is understood that a single crystal form can exist in a variety of different forms (e.g., orthorhombic, monoclinic, triclinic) based on the conditions used for crystallization of the solid but that only one of these crystalline forms is present in a single crystal form.

The invention discloses novel acid addition salts of N-{3-[4-(2-methoxyphenyl)-1-piperazinyl]propyl}-7-oxo-5-trifluoromethyl-7H-thieno[3,2-b]pyran-3-carboxamide. The acid addition salts may be prepared in either crystalline or amorphous form. In some instances, the acid addition salts of the invention may occur as a mixture of crystalline and amorphous forms. The crystalline acid addition salts of the invention may occur as a single crystalline polymorphic form or as a mixture of polymorphic forms. The crystalline and amorphous forms can be identified using techniques known in the art, for example, DSC and XRD. The standard limits of detection when using methods such as DSC and standard powder XRD are approximately 1-2%. More sophisticated XRD techniques can reduce this limit to about 0.5% or less (e.g., synchrotron analysis).

In a preferred embodiment of the invention, an acid addition salt is selected from the group consisting of crystalline mesylate, hydrochloride, hydrobromide and besylate salts of N-{3-[4-(2-methoxyphenyl)-1-piperazinyl]propyl}-7-oxo-5-trifluoromethyl-7H-thieno[3,2-b]pyran-3-carboxamide. In a preferred embodiment the aforementioned crystalline acid addition salts are present in a single crystalline form and are suitable for incorporation into pharmaceutical compositions.

The invention also discloses novel solvated and hydrated forms of the crystalline and amorphous acid addition salts of N-{3-[4-(2-methoxyphenyl)-1-piperazinyl]propyl}-7-oxo-5-trifluoromethyl-7H-thieno[3,2-b]pyran-3-carboxamide disclosed herein. Solvated or hydrated forms of the acid addition salts of the invention may be present as mono-, di-, or higher order solvates or hydrates. Solvates and hydrates may be formed as a result of solvents used during the formation of the acid addition salts of the invention, becoming imbedded in the solid lattice structure upon crystallization. Because formation of the solvates and hydrates occurs during the preparation of a the acid addition salt, formation of a particular solvated or hydrated form depends on the conditions and method used to prepare the salt. Preferably the hydrated and solvated forms of the acid addition salts of the invention include pharmaceutically acceptable solvents.

The novel acid addition salts of the invention are preferably synthesized from the free base of N-{3-[4-(2-methoxyphenyl)-1-piperazinyl]propyl}-7-oxo-5-trifluoromethyl-7H-thieno[3,2-b]pyran-3-carboxamide by the addition of the appropriate acid. In one embodiment, the acid addition salts are prepared by adding a solution of an acid dissolved in a suitable solvent to a solution of the free base dissolved in a suitable solvent. Acids may be selected from, for example and without limitation, (i) inorganic acids, such as, without limitation, hydrochloric acid, hydrobromic acid, phosphoric acid and sulfuric acid; (ii) sulfonic acids, such as without limitation methanesulfonic acid, benzenesulfonic acid, toluenesulfonic acid, and naphthalene-1,5,-disulfonic acid; (iii) monocarboxylic acids, such as, without limitation, acetic acid, (+)-L-lactic acid, DL-lactic acid, DL-mandelic acid, gluconic acid, cinnamic acid, salicylic acid, and gentisic acid; (iv) dicarboxylic acids, such as, without limitation, oxalic acid, 2-oxo-glutaric acid, malonic acid, (−)-L-malic acid, mucic acid, (+)-L-tartaric acid, fumaric acid, maleic acid, and terephthalic acid; (v) tricarboxylic acids, such as, without limitation, citric acid; and (vi) aromatic sulfonamides such as, without limitation, saccharin. Preferred acids used in forming the acid addition salts described herein are hydrochloric acid, hydrobromic acid, methanesulfonic acid and benzenesulfonic acid.

After mixing of the free base and the acid, the salt precipitates or crystallizes, or solvent(s) are removed to yield the solid acid addition salt. The solvent may be removed using any method known in the art, such as evaporation or filtration. The resulting solid acid addition salt may be subjected to crystallization using techniques well known in the art, such as dissolving the solid acid addition salt with water, or a polar protic solvent, such as acetone or an alcohol, and allowing the solvent to slowly evaporate, yielding the crystalline acid addition salt.

Upon formation, the crystalline acid addition salts may be purified, e.g., by recrystallization. In one embodiment, recrystallization is performed in at least two successive steps using polar protic and polar aprotic or non-polar solvents. For example, the crude crystalline acid addition salts can be dissolved into a polar protic solvent (e.g., EtOH) to create a saturated solution and a non-polar (e.g., hexanes) or polar aprotic (e.g., Et₂O) solvent added to the solution to reduce the solubility of the salt, thus leading to the deposition of the crystalline compound with enhanced purity. Purification steps may also include dissolving crude crystals with different solvents at different temperatures or recrystallization of previously recrystallized salts from different or mixed solvent systems.

In a preferred embodiment, the acid addition salts of the invention are prepared in substantially pure form, (i.e., at least 95% purity) with little or no residual solvent. Residual solvent may be removed from the acid addition salts of the invention using any method known in the art, such as evaporating the residual solvent in vacuo or heating the crystalline acid addition salt. Preferably, the acid addition salts are prepared such that residual solvent content is from about 0.1 to about 5% by weight (w/w) and more particularly less than about 2% by weight (w/w).

Preferably a method of preparing the acid addition salts reduces or eliminates unwanted by-products and produces crystalline acid addition salts having only a single crystalline form (i.e., without contamination by other polymorphs). In particular it is preferred that a method reduces or eliminates 4-hydroxy-5-((Z)-4,4,4-trifluoro-3-hydroxybut-2-enoyl)thiophene-3-carboxylic acid {3-[4-(2-methoxyphenyl)piperazin-1-yl]propyl}amide (Formula (III)) and acid addition salts thereof that can result from the hydrolysis of the vinyl ether bond of the pyranyl ring.

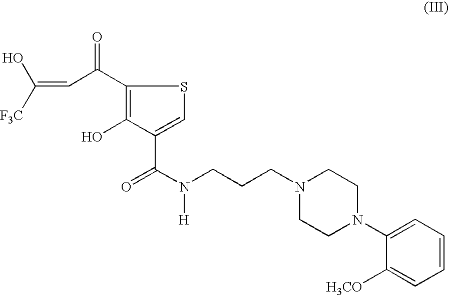

In a preferred embodiment, the method of preparing the acid addition salts results in an acid addition salt of N-{3-[4-(2-methoxyphenyl)-1-piperazinyl]propyl}-7-oxo-5-trifluoromethyl-7H-thieno[3,2-b]pyran-3-carboxamide having an overall purity in the range from about 95 to 99.5% and more preferably in the range from about 96 to about 99.5%, wherein the amount of the compound of formula (III) comprises less then about 2%, more preferably less then about 1% and most preferably less then about 0.5% of the total composition. Hence, in preferred embodiments, the acid addition salts described herein have a purity of, e.g., at least 95%, at least 96%, at least 97%, at least 98%, at least 99%, and at least 99.5%. A preferred method of determining purity of the acid addition salts of the invention is by high performance liquid chromatography.

In one embodiment, the acid addition salts are prepared by reacting N-{3-[4-(2-methoxyphenyl)-1-piperazinyl]propyl}-7-oxo-5-trifluoromethyl-7H-thieno[3,2-b]pyran-3-carboxamide with an acid dissolved in a non-reactive organic solvent. Preferred non-reactive organic solvents include ethanol, dichloromethane, 1,4-dioxane and acetone and mixtures thereof. The formation of the acid addition salt is typically carried out using equimolar amounts of the two reagents, although other ratios are operative. The rate of addition of the acid solution to the free base is not critical to the reaction and the acid may be added rapidly (<5 minutes) or slowly over 6 or more hours. The reaction is carried out at temperatures ranging from about 5 to about 30° C. and more particularly in the range from about 20 to 25° C. Following addition of the acid solution to the free base, the reaction mixture may be stirred until formation of the salt is complete.

The resulting acid addition salts may be isolated from the reaction mixture using any method known in the art, such as by filtration. The yield of the acid salt is generally in the range from about 80% to about 99%. Recrystallization and other purification techniques known in the art may be used to purify the salt further if desired.

The physical and chemical properties of the acid addition salts described herein are distinct from both from one another and from the corresponding free base. For example, compared to crystalline forms of the free base, the acid addition salts of N-{3-[4-(2-methoxyphenyl)-1-piperazinyl]propyl}-7-oxo-5-trifluoromethyl-7H-thieno[3,2-b]pyran-3-carboxamide display greater water solubility. Generally the water solubility of the acid addition salts is from about 1% to about 15%, significantly greater than that of the free base. The acid salts also have X-ray diffraction, NMR, and IR spectra that are distinct from the free base. The physical and chemical properties of the acid addition salts disclosed herein, are discussed in detail below.

The X-ray diffraction pattern of crystalline acid addition salts can be determined using a Philips PW 1710 and Philips X pert PW 3040 powder diffractometer (Copper Kα radiation) under the following typical conditions: About 5-70 mg sample (without any previous treatment) with application of a slight pressure to obtain a flat surface. Ambient air atmosphere. 0.02° 2θ stepwise, 2 sec step-1, 2-50 2θ. One skilled in the art will recognize that the 2θ values will generally be reproducible to within a range from about ±0.10 to about ±0.20 degrees, while the relative intensity of individuals peaks may vary from sample to sample. See e.g., United States Pharmacopoeia XXV (2002), pages 2088-2089.

The crystalline addition salts of the invention exhibit enhanced stability as compared to the corresponding freebase.

In a particular embodiment of the present invention is a crystalline mesylate salt characterized by an off-white-grey color, and a solubility in water at 25° C. ranging from about 5% to about 15% and more specifically from about 8 to about 12%. The mesylate salt preferably exists in a single crystal form (i.e., contains no more than 1-2% of another polymorph), having a melting point as determined by DSC within the range of from about 184° C. to about 188° C., more specifically, about 186° C. The crystalline mesylate salt contains from about 0.01 to about 3.0% of the ring opened impurity of Formula III, preferably from about 0.1 to about 1.5%, and most preferably from about 0.1 to about 1.0%.

In another particular embodiment of the present invention is a crystalline besylate salt characterized by a off-white color, and a solubility in water at 25° C. ranging from about 0.05% to about 0.8% and more specifically from about 0.1% to about 0.3%. The besylate salt can exist in one or more polymorphic forms. The melting point of crystalline besylate polymorphs were determined by DSC. The crystalline besylate salt contains from about 0.01 to about 5% of the ring opened impurity of Formula III, preferably from about 0.1 to about 1.5%, and most preferably from about 0.1 to about 1%.

In yet another particular embodiment of the present invention is a crystalline hydrobromide salt characterized by a off-white color, and a solubility in water at 25° C. ranging from about 0.05% to about 1% and more specifically from about 0.2% to about 0.7%. The hydrobromide salt preferably exists in a single crystal form (i.e., contains no more than 1-2% of another polymorph). The crystalline hydrobromide salt contains from about 0.01 to about 5% of the ring opened impurity of Formula III, preferably from about 0.1 to about 1.5%, and most preferably from about 0.1 to about 1%.

In a further particular embodiment of the present invention is a crystalline hydrochloride salt characterized by a off-white to grey color, and a solubility in water at 25° C. ranging from about 0.1% to about 5% and more specifically from about 0.5% to about 3%. The hydrochloride salt can exist in one or more polymorphic forms. The melting point of crystalline hydrochloride polymorphs, as determined by DSC, are within the range of about 110° C. to about 230° C., more specifically, the DSC shows melting points for two different polymorphs at about 204 or 220° C. and about 113 or 162 or 176° C. (in both cases a mixture of polymorphs). The crystalline hydrochloride salt contains from about 0.01 to about 5% of the ring opened impurity of Formula III, preferably from about 0.1 to about 1.5%, and most preferably from about 0.1 to about 1%.

The most preferred salts of the present invention are those that are synthesized from the free base, contain less than about 2% by weight of the impurity of Formula III and salts thereof, and are present in a single crystal form and not as mixtures of polymorphs (i.e., do not display polymorphism). Thus, the most preferred salt of the present invention is the mesylate salt of N-{3-[4-(2-methoxyphenyl)-1-piperazinyl]propyl}-7-oxo-5-trifluoromethyl-7H-thieno[3,2-b]pyran-3-carboxamide.

The present application further discloses pharmaceutical formulations and unit dosage forms that comprise one of the isolated crystalline acid addition salts of the invention or a mixture thereof.

Pharmaceutical Compositions

The compounds of the invention may be formulated into pharmaceutical compositions. A pharmaceutical composition according to the invention also may include optional excipients or additives, such as a pharmaceutically acceptable carrier or diluent, a flavorant, a sweetener, a preservative, a dye, a binder, a suspending agent, a dispersing agent, a colorant, a disintegrant, an excipient, a film forming agent, a lubricant, a plasticizer, an edible oil or any combination of two or more of the foregoing.

Suitable pharmaceutically acceptable carriers or diluents include, but are not limited to, ethanol; water; glycerol; propylene glycol, aloe vera gel; allantoin; glycerin; vitamin A and E oils; mineral oil; PPG2 myristyl propionate; magnesium carbonate; potassium phosphate; vegetable oil; animal oil; and solketal.

Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; hydroxypropylmethylcellulose; polyethylene glycol; povidone; waxes; and the like. Preferred binders are lactose, hydroxypropylmethylcellulose and povidone.

Suitable disintegrants include, but are not limited to, starch (e.g., corn starch or modified starch) methyl cellulose, agar, bentonite, xanthan gum, sodium starch glycolate, crosspovidone and the like. A preferred disintegrant is sodium starch glycolate.

Suitable lubricants include, but are not limited to, sodium oleate, sodium stearate, sodium stearyl fumarate, magnesium stearate, sodium benzoate, sodium acetate, sodium chloride and the like. A preferred lubricant is magnesium stearate.

Suitable suspending agents include, but are not limited to, bentonite, ethoxylated isostearyl alcohols, polyoxyethylene sorbitol and sorbitan esters, microcrystalline cellulose, aluminum metahydroxide, agar-agar and tragacanth, or mixtures of two or more of these substances, and the like. A preferred suspending agent is microcrystalline cellulose.

Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone and gelatin.

Suitable film forming agents include, but are not limited to, hydroxypropylmethylcellulose, ethylcellulose and polymethacrylates.

Suitable plasticizers include, but are not limited to, polyethylene glycols of different molecular weights (e.g., 200-8000 Da) and propylene glycol. Preferred is polyethylene glycol 6000.

Suitable colorants include, but are not limited to, ferric oxide(s), titanium dioxide and natural and synthetic lacquers. Preferred are ferric oxides and titanium dioxide.

Suitable edible oils include, but are not limited to, cottonseed oil, sesame oil, coconut oil and peanut oil.

Examples of additional additives include, but are not limited to, sorbitol, talc, stearic acid, dicalcium phosphate and polydextrose.

Unit Dosage Forms

The pharmaceutical composition may be formulated as unit dosage forms, such as tablets, pills, capsules, caplets, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. Unit dosage forms may be used for oral, parenteral, intranasal, sublingual or rectal administration, or for administration by transdermal patches. In general, any delivery of active ingredients that results in systemic availability of them can be used. Preferably the unit dosage form is an oral dosage form, most preferably a solid oral dosage form, therefore the preferred dosage forms are tablets, pills, caplets and capsules. However, parenteral preparations also are preferred, especially under circumstances wherein oral administration is cumbersome or impossible.

Preferably the effective oral, parenteral or intravenous dose ranges for human hosts, expressed in mg/kg of body weight per day for use in obstructive disorders of the lower urinary tract is in the range of about 0.001 to about 20, more preferably in the range of about 0.05 to about 3 and most preferably from about 0.5 to about 2. The most-preferred values refer to oral dosing. Intravenous dosages should be 10 to 100 fold lower. Selective-use dosages, i.e., dosages that are active in the lower urinary tract without a substantial effect on blood pressure, depend on the particular compound employed. Generally, in the case of a compound selective in inhibiting urethral contraction, up to four times the amount of the ED₅₀ used in inhibiting urethral contraction can be administered without substantial effect on blood pressure. Further refinements and optimization of dosages are possible using simple routine experiments.

The oral dosage form of the invention preferably contain at least 0.5% of active agent, but the amount of active agent may be varied depending upon the particular form and may conveniently in the range from about 5% to about 70% of the weight of the dosage unit. The amount of active agent in such compositions is such that a suitable dosage will be obtained although the desired dosage can be obtained by administering a plurality of dosage forms. The preferred compositions and preparations according to the invention are prepared so that an oral dosage unit form contains from about 1.0 to about 300 milligrams of active agent.

For the purpose of parenteral therapeutic administration, the active agent may be incorporated into a solution or suspension. These preparations should contain at least 0.1% of active, but it may be varied between 0.5 and about 30% of the weight thereof. The amount of active agent in such compositions is such that a suitable dosage will be obtained. The preferred compositions and preparations according to the invention are prepared so that a parenteral dosage unit contains between 0.2 to 100 milligrams of active agent.

Solid unit dosage forms may be prepared by mixing the active agents of the invention with a pharmaceutically acceptable carrier and any other desired additives as described above. The active ingredient and excipients are typically mixed until a homogeneous mixture is formed, i.e., until the active agents are dispersed evenly throughout the composition. The compositions can then be formed as dry or moist granules, and optionally compressed into tablets, used to fill capsules, etc.

Tablets or pills can be coated or otherwise compounded to form a unit dosage form which has optionally, a modified release profile. For example, the tablet or pill can comprise an inner dosage and an outer dosage component, the latter being in the form of a layer or envelope over the former. The two components can be separated by a release modifying layer which serves to permit dissolution of the active ingredient from the core component over a prolonged period of time. Alternatively, the lease modifying agent is a slowly disintegrating matrix. Additional modified release formulations will be apparent to those skilled in the art.

Biodegradable polymers for controlling the release of the active agents, include, but are not limited to, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

For liquid dosage forms, the active substances or their physiologically acceptable salts are brought into solution, suspension or emulsion, optionally with the usually employed substances such as solubilizers, emulsifiers or other auxiliaries. Solvents for the active combinations and the corresponding physiologically acceptable salts, can include water, physiological salt solutions or alcohols, e.g. ethanol, propanediol or glycerol. Additionally, sugar solutions such as glucose or mannitol solutions may be used. A mixture of the various solvents mentioned may further be used in the invention.

A transdermal dosage form also is contemplated by the invention. Transdermal forms may be a diffusion-driven transdermal system (transdermal patch) using either a fluid reservoir or a drug-in-adhesive matrix system. Other transdermal dosage forms include, but are not limited to, topical gels, lotions, ointments, transmucosal systems and devices, and iontohoretic (electrical diffusion) delivery system. Transdermal dosage forms may be used for timed release and sustained release of the active agents of the invention.

Pharmaceutical compositions and unit dosage forms of the invention for administration parenterally, and in particular by injection, typically include a pharmaceutically acceptable carrier, as described above. A preferred liquid carrier is vegetable oil. Injection may be, for example, intravenous, intrathecal, intramuscular, intraruminal, intratracheal, or subcutaneous.

The active agent also can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The acid addition salts of the invention also may be coupled with soluble polymers as targetable drug carriers. Such polymers include, but are not limited to, polyvinyl-pyrrolidone, pyran copolymer, polyhydroxypropylmethacryl-amidephenol, polyhydroxyethylaspartamidephenol, and polyethyleneoxideopolylysine substituted with palmitoyl residues.

Treatment of Obstructive Syndromes of the Lower Urinary Tract

The compounds and compositions of the invention are useful in the treatment of obstructive syndromes of the lower urinary tract, including benign prostatic hyperplasia (BPH), and of lower urinary tract symptoms (LUTS) as well as of neurogenic lower urinary tract dysfunction (NLUTD), using the novel salts disclosed herein.

In one embodiment, the acid addition salts of the invention may be administered to block α-adrenoceptors and control prostatic smooth-muscle tone. Preferably, administration of the acid addition salts of the invention effectively inhibit contractions of the urethra of the lower urinary tract and relieves lower urinary tract symptoms (LUTS), both of irritative and obstructive type, in patients with BPH. In a preferred embodiment, administration of the acid addition salts of the invention antagonizes the α₁-adrenoceptor and reduces dysfunction of the bladder neck and urethra, thereby decreasing functional outlet obstruction.

In another embodiment, the acid addition salts of the invention may be administered to treat neurogenic lower urinary tract dysfunction (NLUTD), as can be caused by neurological disease or trauma. NLUTD may lead to debilitating symptoms and serious complications, including increased urinary frequency, incontinence, voiding difficulty, recurrent upper urinary tract infections, and upper urinary tract deterioration. Preferably, administration of the acid addition salts of the invention preserve renal function and avoid urological complications. Without being bound by any particular theory, administration of the acid addition salts of the invention may benefit patients with NLUTD by facilitating urine storage by alleviating high detrusor pressure during bladder filling, which is evidenced by poor bladder compliance and detrusor hyperreflexia.

In another embodiment the invention provides for combination therapies to treat BPH, and other lower urinary tract symptoms, and neurogenic lower urinary tract dysfunction. One particular combination comprises an anticholinergic compound, such as oxybutynin, tolterodine, darifenacin, esoxybutynin, solifenacin, imidafenacin, fesoterodine, trospium chloride, and an acid addition salt of the invention. The combination may be administered as separate dosage forms or as unitary dosage form, optionally co-administered with the acid addition salts of the present invention.

The amount of acid addition salt to be administered to treat syndromes of the lower urinary tract is generally a therapeutically effective amount. For example, the amount can be that effective to block an α₁ receptor of an animal, preferably a human or to selectively prevent contractions of the urethra of the lower urinary tract. This amount may vary with the age, size, sex and condition of the animal to be treated, the nature and severity of the disorder to be treated. The total amount of the acid addition salt to be used can be determined by methods known to those skilled in the art. According to one embodiment, from about 0.01 mg/kg to about 10 mg/kg animal body weight (from about 1 to about 700 mg/day), from about 0.03 mg/kg to about 3 mg/kg body weight (from about 2 to about 200 mg/day) or from about 0.1 mg/kg to about 1 mg/kg body weight (from about 5 to about 70 mg/day) of the acid addition salt is administered to treat a specific condition.

### EXAMPLES

The following examples of preparation of acid addition salts of N-{3-[4-(2-methoxyphenyl)-1-piperazinyl]propyl}-7-oxo-5-trifluoromethyl-7H-thieno[3,2b]pyran-3-carboxamide, are now disclosed for illustrative, non-limiting purposes.

### Example 1

### Preparation of the free base of N-{3-[4-(2-Methoxyphenyl)-1-piperazinyl]propyl}-7-oxo-5-trifluoromethyl-7H-thieno[3,2-b]pyran-3-carboxamide free base

The compound of Example 1 was prepared using the methods described in U.S. Pat. No. 6,486,163 the entire contents of which are hereby incorporated by reference. (See e.g., Example 4)

### Example 2

### Preparation of the crystalline mesylate salt of N-{3-[4-(2-methoxyphenyl)-1-piperazinyl]propyl}-7-oxo-5-trifluoromethyl-7H-thieno[3,2-b]pyran-3-carboxamide

2A: Preparation of Crystalline Mesylate Salt in Acetone

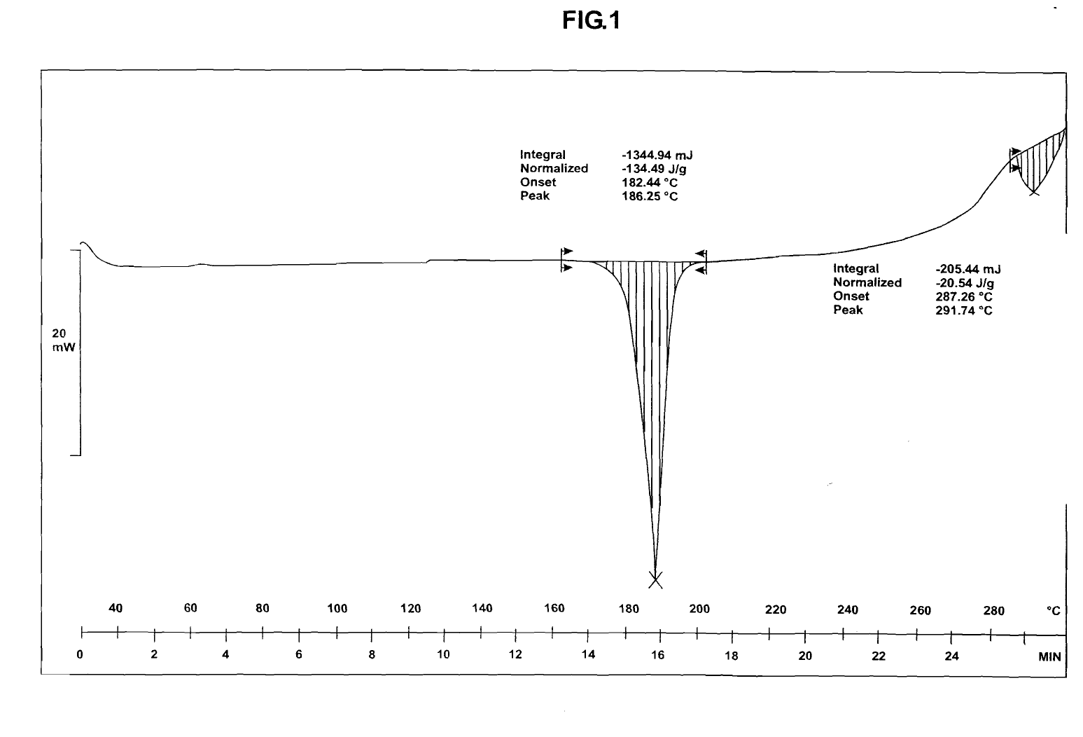

A suspension of the free base was prepared by dissolving 9.92 g (20 mmol) of N-{3-[4-(2-methoxyphenyl)-1-piperazinyl]propyl}-7-oxo-5-trifluoromethyl-7H-thieno[3,2-b]pyran-3-carboxamide free base, prepared as described in Example 1, in 160 mL of acetone. Methanesulfonic acid (2.12 g, 22 mmol) was then rapidly added to the free base suspension at room temperature resulting in dissolution of the free base starting material. Crystallization of the crude free base product occurred upon stirring. The resulting mixture was stirred for 1 hour at room temperature, then allowed to stand at 5° C. for 2 hours. Solid material was separated by filtration and then washed with acetone at 0° C. The washed solid material was then suspended in 100 mL of Et₂O, stirred at room temperature for 30 minutes and collected by filtration. The solid material was dried for 3 hours at 50° C. under vacuum (10 mm Hg) to yield the mesylate salt as an off-white to grey sold. Yield: 11.34 g (95.8%); Purity: 97.6% (determined by (HPLC)); Melting Point: 186° C. (DSC; See FIG. 1); Water Solubility: 100 mg/mL at 25° C.; Residual Solvent: 1.24% (w/w) Acetone; Formula III Impurity: 1.83% (purity, solvent and impurity percents were based on area percents taken from HPLC analysis).

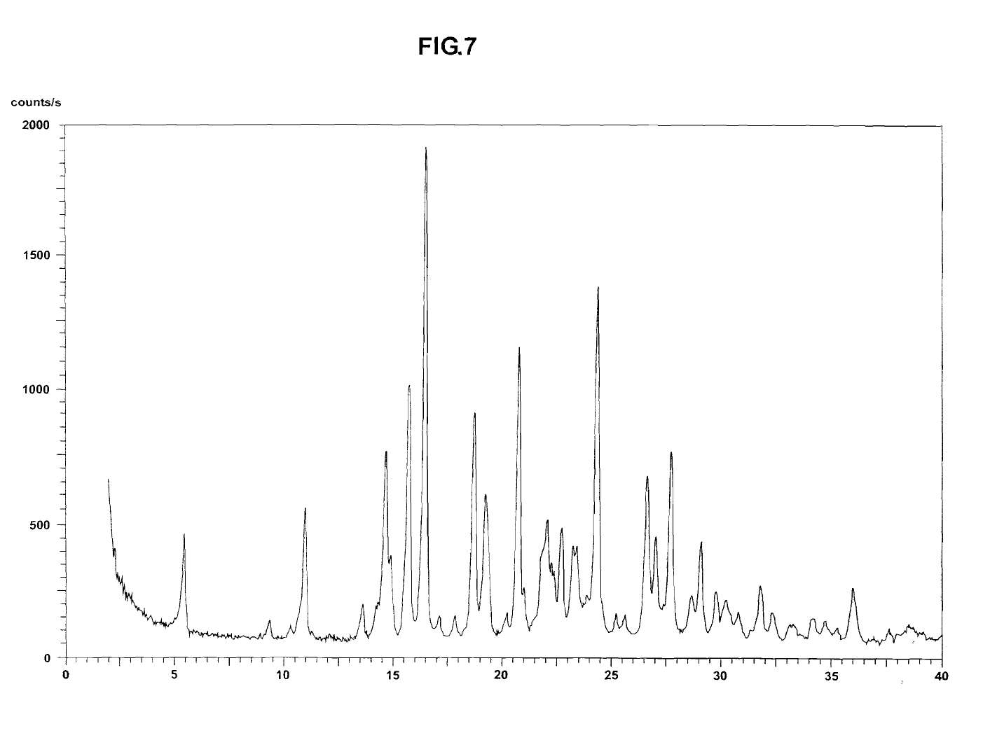

The X-ray diffraction pattern of the crystalline mesylate salt was obtained using a Philips PW 1710 and Philips X pert PW 3040 powder diffractometer (Copper Kα radiation) under the following typical conditions: About 5-70 mg sample (without any previous treatment) with application of a slight pressure to obtain a flat surface. Ambient air atmosphere. 0.02° 2θ stepwise, 2 sec step-1, 2-50 2θ. The XRD spectra is shown in FIG. 7 and a tabulation of the significant XRD peaks is shown in Table 1. XRD peaks are significant if the have a relative intensity (I/I₀) of greater than or equal to 25.0.

TABLE 1
XRD spectrum of N-{3-[4-(2-methoxyphenyl)-1-piperazinyl]propyl}-
7-oxo-5-trifluoromethyl-7H-thieno[3,2-b]pyran-3-carboxamide
mesylate DSC m.p. 186° C.

| D (A) | Relative intensity (I/I₀) | 2θ angle |
|---|---|---|
| 8.1 | 28.8 | 10.97 |
| 6.0 | 39.7 | 14.67 |
| 5.63 | 53.6 | 15.73 |
| 5.37 | 100 | 16.49 |
| 4.73 | 48.5 | 18.75 |
| 4.6 | 32.5 | 19.27 |
| 4.27 | 62 | 20.76 |
| 4.02 | 27 | 22.07 |
| 3.91 | 26.4 | 22.73 |
| 3.65 | 72.8 | 24.35 |
| 3.34 | 35 | 26.65 |
| 3.21 | 43.4 | 27.74 |

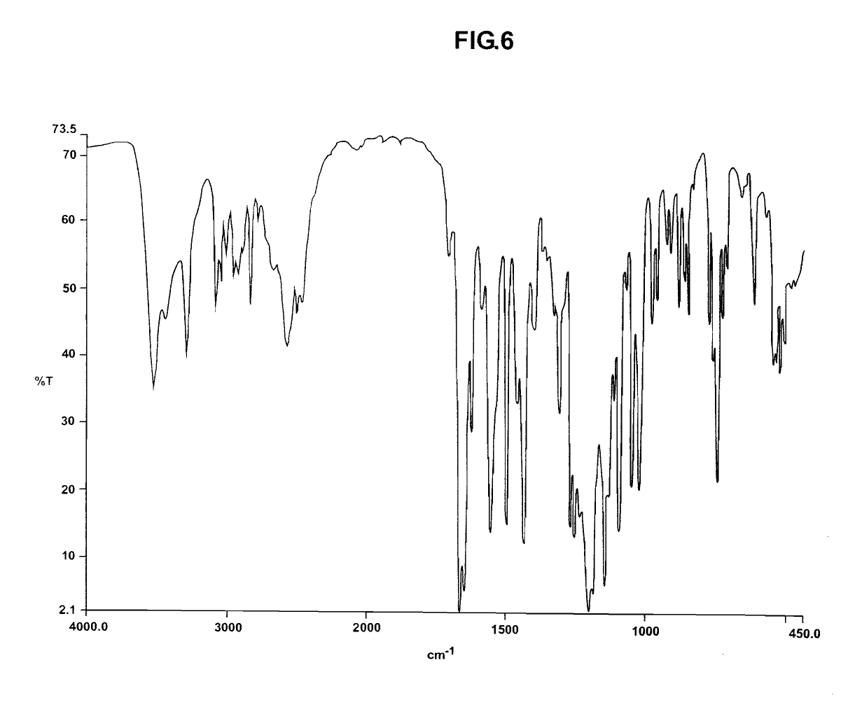

The IR spectrum is shown in FIG. 6. The IR spectra were recorded in KBr pellets on a Perkin-Elmer FT-IR Spectrum One spectrophotometer. Sample preparation: 1 mg of product is mixed thoroughly with 100 mg of dry KBr, then molded as a pellet under 10 tons pressure for at least 15 minutes. 8 scans of spectral data are collected from 450 to 4000 cm⁻¹ and averaged. Results are expressed in % transmittance.

2B: Preparation of Crystalline Mesylate Salt in CH₂Cl₂

A solution of the free base was prepared by dissolving 100 mg (0.2 mmol) of N-{3-[4-(2-methoxyphenyl)-1-piperazinyl]propyl}-7-oxo-5-trifluoromethyl-7H-thieno[3,2-b]pyran-3-carboxamide free base, prepared as described in Example 1 in 2 mL of CH₂Cl₂. Methanesulfonic acid (21.3 mg, 0.22 mmol) was then added to the solution at room temperature. The solution was evaporated to dryness, the crude product suspended in Et₂O and the solid collected by filtration. The crude solid was crystallized from hot acetone to yield 100 mg of the mesylate salt. Yield: 11.34 g (85%); purity: 97.6% (determined by HPLC); melting point: 187° C. (DSC); water solubility: 100 mg/mL at 25° C.; residual solvent: 1.2% (w/w) Acetone; Formula III Impurity: 0.9%.

2C: Recrystallization of Crystalline Mesylate Salt

The crystalline mesylate salt, prepared as described in Example 2A was recrystallized to reduce the amount of impurities. 100 mg of the crystalline mesylate salt was dissolved by heating the mixture with the appropriate solvent (as described in Table 2, below). In the case of water the solvent was allowed to evaporate at room temperature over the course of 72 hours. The final yields of crystalline mesylate salt and the amount of impurities obtained from recrystallization in each solvent, are shown in Table 2. LC purity was calculated by dividing the area under the mesylate salt peak by the total area under all LC peaks.

TABLE 2
Recrystallization of the Mesylate Salt of
N-{3-[4-(2-methoxyphenyl)-1-piperazinyl]propyl}-7-oxo-
5-trifluoromethyl-7H-thieno[3,2-b]pyran-3-carboxamide

| | | Yield of | | |
|---|---|---|---|---|
| | | Crystalline | | Impurity of |
| | Solvent Amount | Mesylate | LC Purity | formula III |
| Solvent | (mL) | (mg) | (area %) | (area %) |
| 99% EtOH | 1.2 | 87 | 98.6 | 1.1 |
| H₂O | 0.25 | 50 | 98.6 | 0.67 |
| 95% Acetone | 2.6 | 70 | 98.5 | 0.8 |
| 95% EtOH | 0.4 | 77 | 98.3 | 1.1 |
| i-PrOH | 2.2 | 88 | 97.8 | 1.3 |

2D: Removal of Residual Acetone by Drying

Residual acetone was removed from crystalline mesylate salts by drying. For example, 100 mg of crystalline mesylate salt (prepared as described in Example 2A) was dried for 4 hours at 85° C. under of vacuum(2 mm Hg), to yield 98 mg of the salt, having 1.04% (w/w) of acetone. In another example, 59 mg of crystalline mesylate salt (prepared as described in Example 2A) was dried for 4 hours at 110° C. under vacuum (2 mm Hg), to yield 58 mg of the salt, having 0.9% (w/w) acetone. In another example, 39 mg of crystalline mesylate salt (prepared as described in Example 2A) was dried for 4 hours at 150° C. under vacuum (2 mm Hg). The final yield of crystalline mesylate salt was 38 mg, having 0.32% (w/w) acetone.

### Example 3

### Preparation of the crystalline hydrochloride salt of N-{3-[4-(2-methoxyphenyl)-1-piperazinyl]propyl}-7-oxo-5-trifluoromethyl-7H-thieno[3,2-b]pyran-3-carboxamide

The hydrochloride salt of N-{3-[4-(2-methoxyphenyl)-1-piperazinyl]propyl}-7-oxo-5-trifluoromethyl-7H-thieno[3,2-b]pyran-3-carboxamide was prepared using the following protocol:

The free base (10.9 g, prepared as described in Example 1) was dissolved in CH₂Cl₂ (250 mL), followed by the addition of 21.1 mL of 1.25 M ethanolic hydrogen chloride solution. The resulting mixture was stirred for 1 hour at room temperature, then the solvents removed in vacuo. The brownish residue was treated with Et₂O (100 mL×3) and the off-white solid was collected by filtration and dried 4 hours (100° C. under vacuum (10 mm Hg)) yielding 10.6 g of the hydrochloride salt.

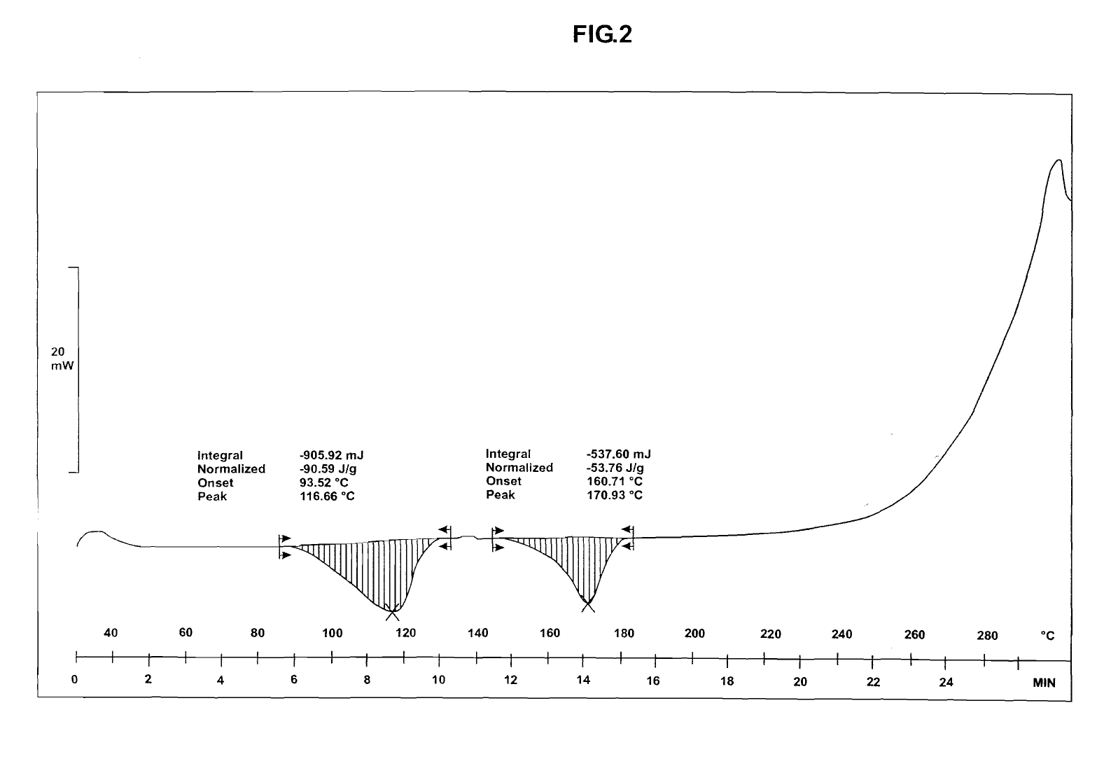

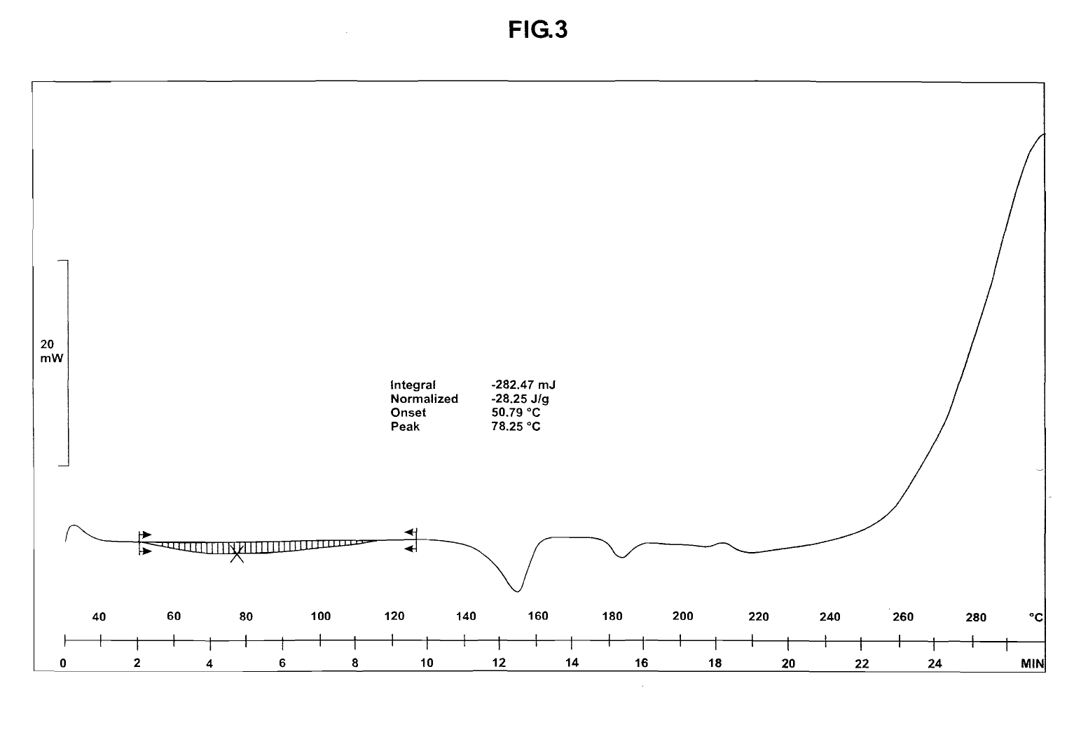

The hydrochloride salt was recrystallized by dissolution in MeOH, followed by the addition of Et₂O. The first crystallized salt was collected (2.14 g, purity 99.7%; DSC (204° C. or 220° C.; See FIG. 2)) followed by the second one (5.75 g, purity 99.8%; DSC (113° C. or 162° C. or 176° C.; See FIG. 3)), for a total amount of 7.89 g (74.4%).

### Example 4

Additional Salt Screening

A salt screening was performed in order to investigate new acid addition salts that displayed high levels of purity, as well as favorable physical characteristics such as salts that crystallized in a single crystalline form. The screening followed the general method below:
1) The free base of Ex. 1 was crystallized from EtOAc, yielding the crystalline free base (88.3%, m.p. 186° C. (DSC) LC 96.5% 3.1% compd III);
2) The addition salts were prepared by either:
   A) The addition of 1 equivalent of the appropriate acid to 0.2 mmol of the free base dissolved in a 9:1 CH₂Cl₂:EtOH solvent system (mL 1.6) at room temperature, followed by removal of the solvent by vacuum evaporation to yield the crude acid addition salt. Recrystallization of the crude acid addition salt from ethanol (mL 1.5).
   B) The addition of 1 equivalent of the appropriate acid to 0.2 mmol of the free base suspended in EtOH (mL 1.6) at room temperature, followed by crystallization of the addition salt product directly from the reaction solution kept at 5° C. for 2 h.

The yield, melting point (by DSC analysis) and purity (by HPLC), for the hydrochloride salt prepared by this method are shown in Table 4. HPLC purity was calculated by dividing the area under the desired salt peak by the total area under all HPLC peaks.

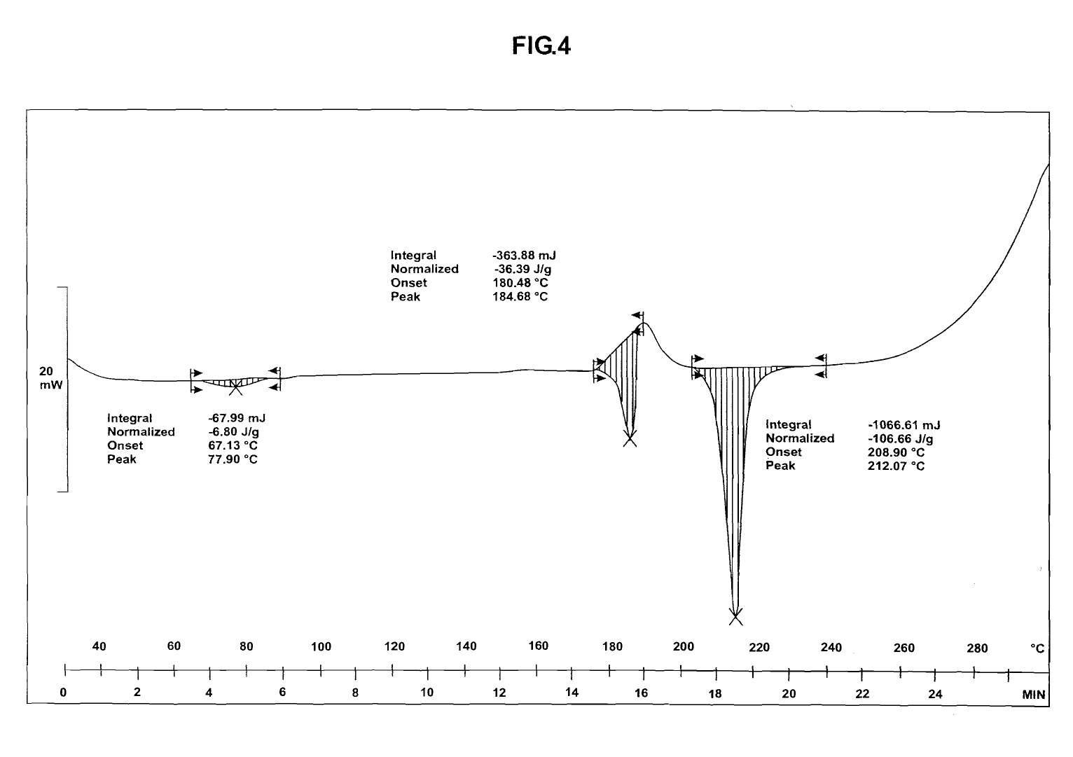

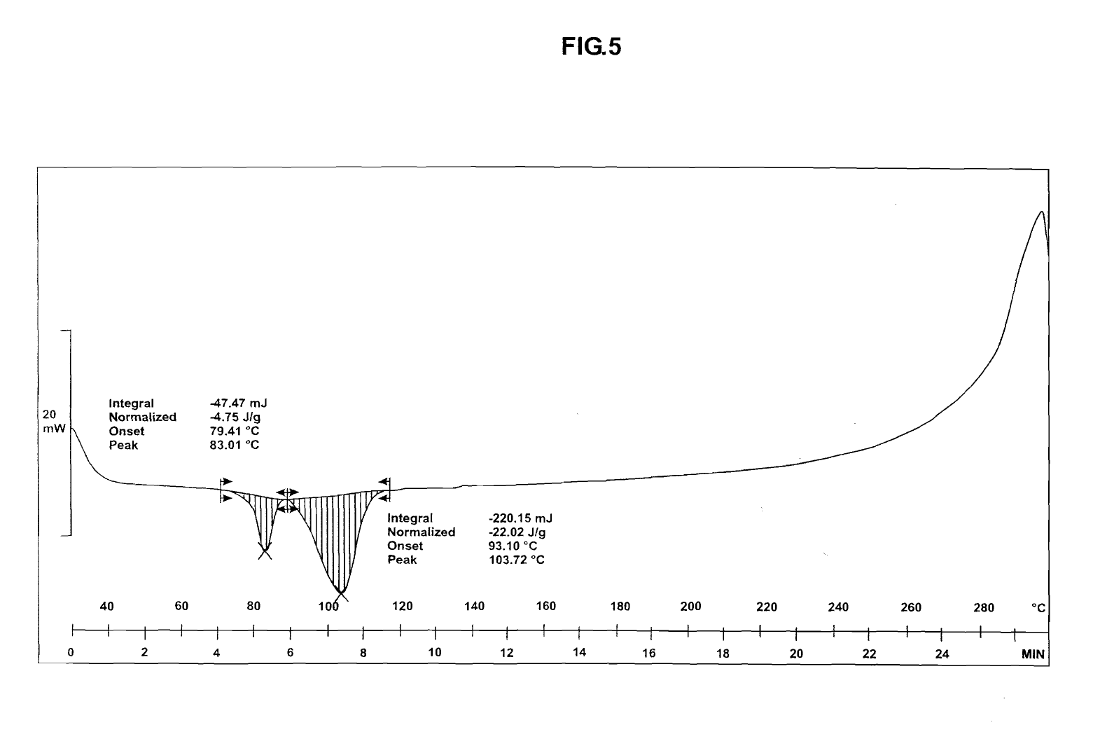

TABLE 4
Properties of crystalline salts

| | Condition | DSC Melting | Purity | Impurity of |
|---|---|---|---|---|
| | (Step 2A vs. | Point(s) | (% by | formula III |
| Salt | Step 2B) | (° C.) | HPLC) | (% by HPLC) |
| HBr | 2A (crude) | 179, 204 | 96.5 | 1.3 |
| | 2A (recrystallized) | 78, 184, 212 | 99 | 0.2 |
| | | (See FIG. 4) | | |
| | 2B | 87, 210, 270 | 96.8 | 1.0 |
| Besylate | 2A (crude) | 80, 100, 146 | 96.2 | 1.04 |
| | 2B | 83, 103 | 96.9 | 0.3 |
| | | (See FIG. 5) | | |

All patents, applications, articles, statutes, and publications mentioned above are hereby incorporated by reference.

The foregoing examples are illustrative only and are not intended to limit the invention in any way. Many variations of the invention will suggest themselves to those skilled in the art in light of the above detailed description. Such obvious variations are within the full intended scope of the appended claims.

We claim:

1. An acid addition salt of N-{3-[4-(2-methoxyphenyl)-1-piperazinyl]propyl}-7-oxo-5-trifluoromethyl-7H-thieno[3,2-b]pyran-3-carboxamide selected from the group of consisting of inorganic, sulfonic, monocarboxylic, and aromatic addition salts of N-{3-[4-(2-methoxyphenyl)-1-piperazinyl]propyl}-7-oxo-5-trifluoromethyl-7H-thieno[3,2-b]pyran-3-carboxamide.

2. The acid addition salt of claim 1 selected from the group consisting of the methanesulfonic, benzenesulfonic, toluenesulfonic, and napthalene-1,5-disulfonic acid addition salts of N-{3-[4-(2-methoxyphenyl)-1-piperazinyl]propyl}-7-oxo-5-trifluoromethyl-7H-thieno[3,2-b]pyran-3-carboxamide.

3. The acid addition salt of claim 1 selected from the group consisting of the hydrochloric, and hydrobromic acid addition salts of N-{3-[4-(2-methoxyphenyl)-1-piperazinyl]propyl}-7-oxo-5-trifluoromethyl-7H-thieno[3,2-b]pyran-3-carboxamide.

4. The acid addition salt of claim 1, wherein the acid addition is salt is the hydrochloric acid addition salt of N-{3-[4-(2-methoxyphenyl)-1-piperazinyl]propyl}-7-oxo-5-trifluoromethyl-7H-thieno[3,2-b]pyran-3-carboxamide.

5. The acid addition salt of claim 1, wherein the acid addition is salt is the methanesulfonic acid addition salt of N-{3-[4-(2-methoxyphenyl)-1-piperazinyl]propyl}-7-oxo-5-trifluoromethyl-7H-thieno[3,2-b]pyran-3-carboxamide.

6. The acid addition salt of claim 1, wherein the acid addition is salt is the hydrobromic acid addition salt of N-{3-[4-(2-methoxyphenyl)-1-piperazinyl]propyl}-7-oxo-5-trifluoromethyl-7H-thieno[3,2-b]pyran-3-carboxamide.

7. The acid addition salt of claim 1, wherein the acid addition is salt is the benzenesulfonic acid addition salt of N-{3-[4-(2-methoxyphenyl)-1-piperazinyl]propyl}-7-oxo-5-trifluoromethyl-7H-thieno[3,2-b]pyran-3-carboxamide.

8. The acid addition salt of claim 1, wherein the salt is in crystalline form and has a water content incorporated into the crystalline lattice of less than 2% (w/w).

9. The acid addition salt of claim 1, wherein said salt is in crystalline form and has a residual solvent content incorporated into the crystalline lattice of less than 3% (w/w).

10. The acid addition salt of claim 1 in the form of a solvate.

11. The acid addition salt of claim 1 in the form of a hydrate.

12. The acid addition salt of claim 1, wherein said salt is substantially pure.

13. The acid addition salt of claim 12, comprising less than about 2% of the compound of formula (III):

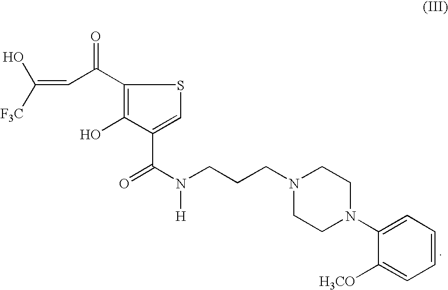

14. A crystalline mesylate salt of N-{3-[4-(2-Methoxyphenyl)-1-piperazinyl]propyl}-7-oxo-5-trifluoromethyl-7H-thieno[3,2-b]pyran-3-carboxamide, having at least one significant x-ray powder diffraction peak at a 2θ value selected from the group consisting of 10.97, 14.67, 15.73, 16.49, 18.75, 20.76, 22.07, 22.73, 24.35, 26.65, and 27.74.

15. The crystalline mesylate salt of claim 14, having at least two significant x-ray powder diffraction peaks at a 2θ value selected from the group consisting of 10.97, 14.67, 15.73, 16.49, 18.75, 20.76, 22.07, 22.73, 24.35, 26.65, and 27.74.

16. The crystalline mesylate salt of claim 14, having at least three significant x-ray powder diffraction peaks at a 2θ value selected from the group consisting of 10.97, 14.67, 15.73, 16.49, 18.75, 20.76, 22.07, 22.73, 24.35, 26.65, and 27.74

17. The crystalline mesylate salt of claim 14, having at least four significant x-ray diffraction peaks at a 2θ value selected from the group consisting of 10.97, 14.67, 15.73, 16.49, 18.75, 20.76, 22.07, 22.73, 24.35, 26.65, and 27.74.

18. A pharmaceutical compositions comprising the acid addition salt of claim 1 and a pharmaceutically acceptable excipient and/or carrier.

19. The pharmaceutical composition of claim 18, comprising at least one component selected from the group consisting of: (i) a pharmaceutically acceptable diluent, (ii) a flavorant, a sweetener, or a preservative, (iii) a dye, (iv) a binder, (v) a suspending agent, (vi) a dispersing agent, (vii) a colorant, (viii) a disintegrant, (ix) a lubricant, (x) a plasticizer, and (xi) an edible oil.

20. A method for preparing an acid addition salt of N-{3-[4-(2-methoxyphenyl)-1-piperazinyl]propyl}-7-oxo-5-trifluoromethyl-7H-thieno[3,2-b]pyran-3-carboxamide comprising, reacting the free base of N-{3-[4-(2-methoxyphenyl)-1-piperazinyl]propyl}-7-oxo-5-trifluoromethyl-7H-thieno[3,2-b]pyran-3-carboxamide with an acid in an organic solvent to form the acid addition salt.

21. The method of claim 20, wherein the acid is selected from the group consisting of: (i) inorganic acids, and (ii) sulfonic acids.

22. The method of claim 20, further comprising the step of isolating the acid addition salt by removing said organic solvent.

23. The method of claim 22, further comprising recrystallizing the isolated acid addition salt in at least one of two successive steps: (a) dissolving the acid addition salt in a polar protic solvent; and (b) adding a polar aprotic or nonpolar solvent.

24. The method of claim 23, wherein the isolated acid addition salt is recrystallized by dissolving the acid addition salt in a polar protic solvent.

25. The method of claim 23, wherein the isolated acid addition salt is recrystallized by dissolving the acid addition salt in a polar protic solvent and adding a polar aprotic or nonpolar solvent.
